(19) United States
(12) Patent Application Publication
(10) Pub. No.: US 2025/0121107 A1
(43) Pub. Date: Apr. 17, 2025

(54) METHOD FOR STEAM STERILIZATION IN AN AUTOCLAVE AND AUTOCLAVE

(71) Applicant: MELAG Medizintechnik GmbH & Co. KG, Berlin (DE)

(72) Inventors: Eberhard VON DER WAYDBRINK, Paulinenaue (DE); Stephan KÜNKEL, Hoppegarten (DE); Guido LITZBA, Teltow (DE); André BERTHE, Berlin (DE); Tino BECKER, Berlin (DE); Martin HERMANN, Berlin (DE); Philipp SEIBERT, Berlin (DE)

(21) Appl. No.: 18/915,244

(22) Filed: Oct. 14, 2024

(30) Foreign Application Priority Data
Oct. 16, 2023 (EP) 23 203 860.4

(51) Int. Cl. A61L 2/07; A61L 2/26; C02F 1/00; C02F 1/02

(52) U.S. Cl. CPC A61L 2/07; A61L 2/26; C02F 1/001; C02F 1/02; A61L 2202/11; A61L 2202/121; A61L 2202/122; A61L 2202/15; A61L 2202/24; C02F 2303/04

(57) ABSTRACT

It is provided a method for steam sterilization in an autoclave. The method comprises the generation of gaseous water in an instantaneous steam generator, the provision of the gaseous water in a chamber of the autoclave for sterilizing sterile items arranged in the chamber, and the setting of a sterilization condition in the chamber for the duration of a holding time, wherein all of the water present in the chamber during the holding time is sterilized.

### CROSS REFERENCE TO RELATED APPLICATION

The present application claims priority to European Patent Application No. 23 203 860.4 filed on Oct. 16, 2023. The entire contents of the above-listed application are hereby incorporated by reference for all purposes.

### BACKGROUND

The proposed solution relates to a method for steam sterilization in an autoclave and to a corresponding autoclave.

Steam sterilization is used for example to sterilize medical devices. In a sterilization chamber of a steam sterilizer or autoclave steam is utilized in order to reduce the number of active and reproducible microorganisms. To achieve a sufficient reduction of the corresponding microorganisms, the relevant standards provide that a sterilization condition is maintained for the duration of a holding time in the chamber. For example, EN 285 provides that as a sterilization condition a sterilization temperature of 121° C. is maintained over a holding time of 15 minutes or a sterilization temperature of 134° C. is maintained over a holding time of three minutes, each at saturated steam conditions. EN 13060 in addition provides as sterilization conditions a sterilization temperature of 126° C. with a holding time of ten minutes and a sterilization temperature of 143° C. with a holding time of one minute, both likewise at saturated steam conditions. Only when maintaining the sterilization conditions for the duration of the holding time, a sufficient reduction of microorganisms and hence a sterilization is guaranteed. In addition, the holding time ensures the maintenance of the sterilization value.

The steam is generated from water by heating. For generating the steam, so-called instantaneous steam generators also are used to an increasing extent. An instantaneous steam generator instantaneously evaporates the water flowing through the same, so that a steam generation immediately is effected when the regulation in the sterilization chamber, in the following briefly called chamber, provides a steam inlet or a pressure increase. In contrast to ordinary steam generators, the steam is generated neither over an extended period nor is it necessary to keep the same in stock in a reservoir. The steam generation rather is effected on demand. This provides for a faster and more efficient supply of the autoclave with steam at comparatively low costs. An instantaneous steam generator is heated already before supplying feed water, i.e. liquid water. The steam generation then is triggered by supplying feed water. Here, the greater thermal capacity of the instantaneous steam generator as compared to a conventional steam generator is advantageous, which ensures that there is no sharp drop in temperature due to evaporation. In contrast thereto, ordinary steam generators are fed with liquid water, but the steam generation then is only triggered by switching on the heating. The patent document EP 972 159 B1 for example discloses an instantaneous steam generator. Water is passed through a heated metal block and evaporated in the same. The steam thus generated is passed into the chamber of an autoclave in order to here set the chamber pressure and temperature to the values required for sterilization. EP 681 141 A1, DE 69 622 316 and EP 1 516 632 A1 show further variants of instantaneous steam generators.

Here, however, it is disadvantageous that during the holding time new water again and again is evaporated in the instantaneous steam generator and introduced into the chamber in order to here reach the necessary increases in pressure and/or temperature. This steam itself, however, is not subject to the sterilization conditions for the duration of the holding time. Thus, the steam fed into the chamber from the instantaneous steam generator after the beginning of the holding time is not sterile itself and is not sterilized either in the chamber due to the insufficient remaining holding time. Microorganisms present in the steam, in particular in aerosol droplets contained therein, thus are not inactivated sufficiently, so that the steam can contaminate sterile items arranged in the chamber with microorganisms. Thus, no sterile sterilized items are present in the chamber at the end of the holding time. This is even more alarming as the water must be demineralized before evaporation in the steam generator. This can result in longer service lives of the water with the corresponding nucleation and contamination. As microbes possibly present in the water in use of an instantaneous steam generator are not exposed to the sterilization conditions for the duration of the holding time, these microbes consequently are not necessarily rendered harmless, but contaminate the items actually to be sterilized, which are present in the chamber. The patent applications EP 1 623 724 A1 and WO 2009/039 616 A1 describe methods and apparatuses for reducing the biofilm formation in an autoclave, but do not call into question the suitability of water potentially containing microbes for the sterilization.

### SUMMARY

Therefore, it is an object underlying the proposed solution to provide a method for steam sterilization with an autoclave and an autoclave, which provides for a hygienically perfect, i.e. actually sterile, steam sterilization despite an instantaneous steam generation.

The object is achieved by a method for steam sterilization in an autoclave with features as described herein. The method comprises the step of generating gaseous water, also called steam, in an instantaneous steam generator. In an instantaneous steam generator the steam generation is effected instantaneously, for example when passing liquid water through a bore in a heatable block, in particular metal block. In the instantaneous steam generator, gaseous water is generated. The gaseous water is provided in a chamber, in particular a sterilization chamber for accommodating sterile items, of the autoclave for sterilization of sterile items arranged in the chamber. For the duration of a holding time, a sterilization condition in the chamber is set. A sterilization condition for example comprises a sterilization temperature and/or a sterilization pressure. In particular, the sterilization condition comprises a sterilization temperature and a sterilization pressure, wherein the sterilization pressure is the pressure obtained at the sterilization temperature according to the saturated steam table. The holding time in particular is chosen in dependence on the sterilization condition. For example, what is set as a sterilization condition is a sterilization temperature of 121° C. at saturated steam conditions for the duration of a holding time in the chamber, wherein the holding time for this sterilization condition is 15 minutes. Alternatively, what is set as a sterilization condition is a sterilization temperature of 134° C. at saturated steam conditions for the duration of a holding time in the chamber, wherein the holding time for this sterilization condition merely is three minutes. Other sterilization conditions with their corresponding holding times, which are regulated in the relevant standards concerning the steam sterilization, also represent examples for sterilization conditions and holding times in accordance with the solution. The method is characterized in that all of the water present in the chamber during the holding time is sterilized, in particular such that at the end of the holding time exclusively sterilized water is present in the chamber. The term “water” here comprises water both in its gaseous and in its liquid phase, hence both liquid water and steam. Sterilization comprises both steam sterilization and sterile filtration. In accordance with DIN EN 556 the term “sterile” in the following refers to a state in which the theoretical probability of finding a viable microorganism is less than 1:1,000,000. By sterilizing all of the water which is provided in the chamber during the holding time, there is provided a hygienically perfect method for steam sterilization by using an instantaneous steam generator. A contamination of the chamber with non-sterilized water, as it is effected in the prior art during steam sterilization with instantaneous steam generation, is avoided. The sterilization of all of the water provided in the chamber during the holding time is effected before and/or during the provision in the chamber, i.e. before and/or during the holding time, in liquid and/or gaseous phase.

According to one aspect of the method, all of the water present in the chamber during the holding time is sterilized before being provided in the chamber or is sterilized after being provided in the chamber or is partly sterilized before being provided in the chamber and completely sterilized after being provided in the chamber. In particular, all of the water present in the chamber during the holding time is sterilized before the beginning of the holding time or is sterilized during the holding time or is partly sterilized before the holding time and completely sterilized during the holding time. The water for example is completely sterilized when a sterility assurance level (SAL) of 10⁻⁶ is reached, i.e. the theoretical probability of finding a viable microorganism is 1:1,000,000 or less. The SAL value indicates the probability with which a single viable microorganism is to be found on a sterile item after the sterilization. The SAL value is indicated as a negative exponent to the base 10 and is a quantitative value. An SAL value of 10⁻⁶ is a smaller value, but designates a greater assurance of sterility than for example an SAL value of 10⁻¹. The water is partly sterilized in this sense, when an SAL value greater than 10⁻⁶ has been reached. As a minimum temperature for the microbial inactivation a temperature of at least 115° C. is provided in the steam sterilization, see e.g. draft standard ISO/FDIS 17665. From this temperature, an action of water, in particular of gaseous water, is effected in the sense of a sterilization. Under these conditions, water is already partly sterilized. A complete sterilization, however, requires higher temperatures with corresponding holding times. The last alternative thus describes a sterilization of the water in several, in particular two, steps. Both the sterilization and the partial sterilization as well as the complete sterilization refer to water in its gaseous and/or in its liquid phase. By sterilizing the water before provision in the chamber it is ensured that only sterile water at all gets into the chamber, i.e. the same is not contaminated with microorganisms due to the water. When the water is sterilized after being provided in the chamber, it is ensured that microorganisms which possibly have come into the chamber due to the water are inactivated in a sufficient number in order to ensure a sterilization of items stored in the chamber. When the water merely is partly sterilized before being provided in the chamber, a complete sterilization in the chamber is necessary in order to ensure a sterilization of items stored in the chamber.

In one aspect of the method, setting of the sterilization condition during the holding time is effected exclusively by evaporation of condensate obtained in the chamber. It thereby is ensured that all of the water provided in the chamber during the holding time is subject to a sterilization condition for the duration of the holding time. All of the water provided in the chamber during the holding time thus is sterilized in the chamber. In particular, no new water is fed into the chamber during the holding time, which then is subjected to the sterilization condition in the chamber only for a period of time shortened with respect to the holding time and thus would not necessarily be sterile at the end of the holding time. In an alternative aspect of the method, water is fed into the chamber before the beginning of the holding time. Setting of the sterilization condition during the holding time is effected exclusively by evaporation of the water fed into the chamber before the holding time and by evaporation of condensate obtained in the chamber during the holding time. All of the water present in the chamber during the holding time thus is subject to a sterilization condition for the duration of the holding time, thus is sterilized.

According to another aspect of the method, a condensate obtained in the chamber is evaporated in the chamber. In one variant, water fed into the chamber before the beginning of the holding time additionally is evaporated in the chamber. Via the evaporation of the condensate, in particular of the condensate and the water fed into the chamber before the beginning of the holding time, in the chamber a sterilization condition can be set during the holding time, without having to feed new water into the chamber. The evaporation is effected for example by means of a heating element arranged in the chamber. Alternatively, the instantaneous steam generator itself is arranged in the chamber, so that the evaporation is effected by means of the instantaneous steam generator in the chamber. An additional heating element with a corresponding regulation is not needed here. The instantaneous steam generation is effected directly in the chamber, so that the gaseous water can also be provided here directly. In a first variant, the instantaneous steam generator comprises a heating coil arranged in the chamber, in particular a tubular radiator, wherein water fed into the chamber directly contacts the heating coil, in particular the tubular radiator, and thus is evaporated. For example, the autoclave includes a nozzle arranged in the chamber and aligned with the heating coil, in particular the tubular radiator, for directly feeding or spraying liquid water onto the heating coil, in particular the tubular radiator. The feeding of liquid water is effected in particular when the heating coil is switched on, in particular when the tubular radiator is switched on. This distinguishes the instantaneous steam generator from an ordinary steam generator, which does not provide an injection of liquid water directly onto the heating coil, in particular the tubular radiator, and begins heating of the heating coil, in particular of the tubular radiator, only after the feeding operation.

For example, the instantaneous steam generator comprises a radiator, in particular a heating coil or a heating cartridge, wherein the heating coil or the heating cartridge is arranged in a heating block. The heating block is arranged for example in the region of the floor of the chamber. In one variant, the heating block is fabricated by means of aluminum die-casting or aluminum extrusion. For example, the autoclave includes a nozzle arranged in the chamber for feeding, in particular spraying, liquid water onto the radiator. The feeding of liquid water is effected in particular with the radiator switched on. The radiator thus acts as an instantaneous steam generator.

In another variant, the instantaneous steam generator comprises the entire chamber wall or parts of the chamber wall, for example the floor of the chamber and/or the lateral chamber wall. In the chamber wall, in particular in the chamber floor, at least one heating element, in particular a heating cartridge is arranged in one example, by means of which the chamber wall, in particular the chamber floor can be heated and act as an instantaneous steam generator. In addition, the instantaneous steam generator comprises the lateral chamber wall. In this variant, at least one heating element alternatively or additionally is arranged in the lateral chamber wall, by means of which the chamber wall is heated and utilized for instantaneous steam generation. Heating elements in the lateral chamber wall in one variant merely are provided to heat the lateral chamber wall and reduce or inhibit a formation of condensate. For example, the instantaneous steam generator comprises the entire chamber wall or parts of the chamber wall, wherein the chamber is an extruded boiler, in particular an extruded aluminum boiler. Alternatively, the extruded boiler is fabricated from another thermally conductive material, for example from copper. In another example, the chamber is fabricated by a die-casting method, in particular by means of aluminum die-casting.

In another alternative, the instantaneous steam generator is arranged on the chamber wall, in particular on the chamber floor. For example, the instantaneous steam generator comprises induction heating elements. In another variant, the instantaneous steam generator comprises radiator arranged on the outside of the chamber wall, in particular thick-film elements arranged on the outside of the chamber wall.

The chamber for example includes a nozzle via which water can be injected into the chamber. Thus, the water does not directly get in contact with the heated parts of the chamber wall, which act as instantaneous steam generator. Water sprayed onto the chamber wall, in particular the chamber floor, thus can be evaporated instantaneously.

In one variant, the setting of a sterilization condition in the chamber for the duration of a holding time is effected by means of a regulating unit, wherein the regulating unit includes at least one sensor. The sensor for example is a temperature sensor or a pressure sensor. In one variant, the regulating unit comprises at least one temperature sensor, wherein the at least one temperature sensor is arranged in the interior of the chamber or is arranged in contact with the chamber wall in the interior of the chamber or outside the chamber. In one variant, the at least one temperature sensor is arranged on the heating element arranged in the floor of the chamber. Alternatively, the regulating unit includes at least one, in particular exactly one, pressure sensor in the interior of the chamber.

Alternatively, condensate obtained in the chamber, or condensate obtained in the chamber and water fed into the chamber before the beginning of the holding time, is evaporated outside the chamber. For example, the instantaneous steam generator is arranged outside the chamber and the evaporation of the condensate, or of the condensate and the water fed into the chamber before the beginning of the holding time, is effected in the instantaneous steam generator outside the chamber. Instantaneous steam generator and chamber here are separated, but form a circuit for the condensate or for the condensate and the water fed into the chamber before the beginning of the holding time or for the gaseous water generated by its evaporation. An additional heating element is not needed.

According to one aspect, the condensate obtained in the chamber, or the condensate obtained in the chamber and the water fed into the chamber before the beginning of the holding time, is pumped from the chamber into the instantaneous steam generator arranged outside the chamber by means of a pump. Chamber, instantaneous steam generator and pump can form a circuit in which the condensate or the condensate and the water fed into the chamber before the beginning of the holding time or the gaseous water generated therefrom circulates during the holding time as required. For example, a port regularly provided in autoclaves for discharging the condensate from the chamber here can be utilized for supplying the condensate, or the condensate and the water fed into the chamber before the beginning of the holding time, to the instantaneous steam generator. In one example, the pump is a pump especially provided for this circuit. In an alternative example, a pump is utilized for introducing feed water, i.e. liquid water, from a tank into the instantaneous steam generator and for pumping the condensate, or the condensate obtained in the chamber and the water fed into the chamber before the beginning of the holding time, from the chamber to the instantaneous steam generator. For example, there is provided a multi-way valve, such as a 3/2-way valve, in order to connect the two circuits to each other. Thus, only one pump is needed both for introducing feed water into the instantaneous steam generator and for recirculating the condensate, or the condensate obtained in the chamber and the water fed into the chamber before the beginning of the holding time, to the instantaneous steam generator.

According to one aspect, setting of the sterilization condition is effected during the holding time, in particular also by refeeding gaseous water into the chamber. According to this aspect, instead of or in addition to the water provided in the chamber before the beginning of the holding time further water is utilized in order to set the sterilization condition.

For example, the instantaneous steam generator includes a steam reservoir sealed off with respect to the chamber by means of a valve. In the instantaneous steam generator gaseous water is generated in stock and passed into the chamber during the holding time for setting the sterilization condition. Thus, no new feed water is needed during the holding time for setting the sterilization condition, which would not be subject to a sterilization condition for the duration of the holding time. The steam reservoir for example can be sealed off with respect to the chamber via a valve, so that setting of the sterilization condition is effected via adjusting operations at the valve. In one example, to avoid a dead section and a risk of mixing with new feed water in the region of a feed water inlet of the instantaneous steam generator, a valve, in particular a non-return valve, is arranged in this region. Alternative measures to avoid such a dead section and the related risk of mixing are known to the skilled person and can likewise be applied.

According to one aspect, the water is sterilized before being supplied to the instantaneous steam generator. Thus, all of the (liquid) water getting into the instantaneous steam generator already is sterile, so that the steam produced in the instantaneous steam generator no longer needs to be sterilized.

For example, the sterilization of the water is effected in a storage tank disposed upstream of the instantaneous steam generator by heating to a temperature above a sterilization temperature. For this purpose, feed water, e.g. from a feed water tank, is fed into a storage tank. The storage tank in particular has its own heating, by means of which the introduced water is heated to a temperature above a sterilization temperature. For example, the introduced water is heated to 150° C. The storage tank in one variant is designed such that it can store the water volume needed for a sterilization process during the holding time. In one example, a force pilot operated valve is provided between the storage tank and the instantaneous steam generator, so that an overpressure obtained by heating the water to a temperature above a sterilization temperature can be utilized to transfer the preheated water via the force pilot operated valve into the instantaneous steam generator. In one example, to avoid a dead section and a risk of mixing with new feed water in the region of a feed water inlet of the instantaneous steam generator, a valve, in particular a non-return valve, is arranged in this region. Alternative measures to avoid such a dead section and the related risk of mixing are known to the skilled person and can likewise be applied.

Alternatively, the sterilization of the water is effected in a coil arranged in or on the chamber. The coil is configured to store a water volume needed for a sterilization process during the holding time. For example, new feed water is passed over a coil before being fed into the instantaneous steam generator. The coil is arranged in the chamber or on the chamber so that the coil is exposed to water flowing through for the duration of the holding time of the sterilization condition. Thus, it is ensured that all of the water used for sterilization was exposed to the sterilization condition for the duration of the holding time and thus has been sterilized before being provided in the chamber. Alternatively, the water is merely partly sterilized in the coil. For example, the water in the coil is subjected to a minimum temperature for microbial inactivation, e.g. to a minimum temperature of 115° C. From this minimum temperature, the water already is partly sterilized, i.e. the number of active microorganisms is reduced. A complete sterilization, i.e. reaching an SAL value of 10⁻⁶, requires higher temperatures than the minimum temperature for microbial inactivation of for example 115° C. with corresponding holding times. The temperature profile in the coil in one variant corresponds to the temporally delayed course of the temperature in the interior of the chamber. In one variant, the sterilization is completed in the chamber after feeding the steam from the instantaneous steam generator into the chamber, i.e. the steam in the chamber is subjected to a sterilization condition for a sufficient period so that the SAL value of 10⁻⁶ is reached at the end of the holding time. The coil is fed with feed water, e.g. from a tank, for example by means of a pump. In one variant, a force pilot operated valve is arranged between coil and instantaneous steam generator. An automatic further flow of water into the instantaneous steam generator only is possible in a method with pre-vacuum or in a fractionated vacuum method due to the negative pressure in the chamber in dependence on the type of the pump, which can be avoided with a force pilot operated valve. In an alternative variant, the coil is operated in a pure overpressure method, also called flow method, and without a force pilot operated valve. In one example, to avoid a dead section and a risk of mixing with new feed water in the region of a feed water inlet of the coil, a valve, in particular a non-return valve, is arranged in this region. Alternative measures to avoid such a dead section and the related risk of mixing are known to the skilled person and can likewise be applied.

In another alternative, the water is sterilized by means of a sterile filter before being supplied to the instantaneous steam generator. For example, the sterile filtration is effected in a feed line from a tank for feed water to the instantaneous steam generator. Alternatively or additionally, the sterile filtration is effected in the region of an inlet to the instantaneous steam generator. In these two variants liquid water is sterilized by means of sterile filtration. Alternatively or additionally, the gaseous water generated in the instantaneous steam generator is sterilized by means of a sterile filter before being provided in the chamber. For example, a sterile filter is arranged in the region of an outlet of the instantaneous steam generator. Alternatively or additionally, a sterile filter is arranged in the region of the feed line from the instantaneous steam generator to the chamber. In these variants, gaseous water is passed through the sterile filter(s) so that the same is/are sterilized continuously due to the through-flow of steam. Sterile filters arranged in the region of the inlet and the outlet of the instantaneous steam generator also are thermally sterilized continuously due to the intensive action of heat caused by the instantaneous steam generator. In these cases an exchange of the sterile filters only is necessary at the end of the technical lifetime of the sterile filters. An exchange of the sterile filter due to an exhaustion of its hygienic capacity merely is necessary when arranged in a feed line from the feed water tank to the instantaneous steam generator. An arrangement of sterile filters in the region of an outlet of the instantaneous steam generator or in a feed line from the instantaneous steam generator to the chamber also has the further benefit that aerosol droplets are held back by the sterile filter, and the quality of the steam generated by the instantaneous steam generator thus is increased not only in hygienic terms, but also in technical terms.

Another subject-matter of the proposed solution is an autoclave for sterilizing sterile items with features as described herein. The autoclave includes an instantaneous steam generator for generating gaseous water. Furthermore, the autoclave includes a regulating unit for setting a sterilization condition in a chamber of the autoclave for the duration of a holding time. The chamber is suited and provided for accommodating sterile items. The autoclave is characterized in that it is configured to sterilize all of the water present in the chamber during the holding time, in particular such that at the end of the holding time exclusively sterile water is present in the chamber. With the autoclave, the advantages of the method of the solution for steam sterilization likewise are realized. In particular, in the present case there is provided an autoclave comprising an instantaneous steam generator which is hygienically safe, because the water provided in the chamber for sterilization is sterile by the end of the holding time. A contamination of the chamber and of sterile items arranged in the chamber with microorganisms due to the instantaneous steam generation thus is avoided.

The autoclave in particular is configured to carry out all steps described with regard to the method for steam sterilization. Thus, the apparatus features corresponding to the features described with regard to the method also represent optional features of the autoclave, and vice versa.

According to one aspect, the autoclave is configured to sterilize all of the water present in the chamber during the holding time before being provided in the chamber. Alternatively, the autoclave is configured to sterilize all of the water present in the chamber during the holding time after being provided in the chamber. In another alternative, the autoclave is configured to partly sterilize all of the water present in the chamber during the holding time before being provided in the chamber, and to completely sterilize the water after being provided in the chamber. The autoclave for example includes a unit for sterilizing or partly sterilizing the water before being fed into the chamber.

In one variant, the instantaneous steam generator is arranged outside the chamber. The autoclave, in particular the unit for sterilizing or partly sterilizing the water before being fed into the chamber, in this variant includes a heatable storage tank disposed upstream of the instantaneous steam generator for heating water to a temperature above a sterilization temperature. The temperature above the sterilization temperature for example is 150° C. The storage tank for example includes a heater for heating the water in the storage tank. Thus, the storage tank is suited to partly or completely sterilize the water arranged in the same. For example, the storage tank is connected to a tank for feed water. In one variant a pump is provided in the feed line, by means of which feed water can be pumped from the tank into the storage tank. An outlet of the storage tank is connected to an inlet of the instantaneous steam generator. In one variant, a force pilot operated valve is arranged in the feed line from the storage tank to the instantaneous steam generator. Overpressure produced in the storage tank can thus be utilized to transfer the preheated water into the instantaneous steam generator by means of the valve. The storage tank in particular is designed such that it can hold, i.e. store, the water needed for a sterilization program during the holding time. The gaseous water generated in the instantaneous steam generator is provided in the chamber via a feed line.

In one variant, the instantaneous steam generator is arranged outside the chamber, and the autoclave, in particular the unit for sterilizing or partly sterilizing the water before being fed into the chamber, includes a coil disposed upstream of the instantaneous steam generator, which is arranged in or on the chamber, for storing a water volume needed for a sterilization process. In the coil, the water guided through the same is partly sterilized or sterilized. The coil is designed such that it can store the water needed for a sterilization process during a holding time. The coil in one variant is fed with feed water from a tank by means of a pump. The pump is arranged in a feed line between a tank for feed water and the coil. An outlet of the coil is connected to an inlet of the instantaneous steam generator. A force pilot operated valve for example is arranged in the feed line between coil and instantaneous steam generator. The gaseous water generated in the instantaneous steam generator is passed into the chamber via a feed line.

In another variant, the autoclave, in particular the unit for sterilizing or partly sterilizing the water before being fed into the chamber, includes a steam reservoir of the instantaneous steam generator which is sealed off with respect to the chamber by means of a valve. Thus, gaseous water can be generated in stock with and in the instantaneous steam generator. Via an outlet of the instantaneous steam generator, in particular its steam reservoir, the gaseous water can be passed into the chamber via adjusting operations at the valve. Thus, no new feed water is needed during the holding time. During the holding time, the autoclave exclusively operates with the gaseous water stored in the steam reservoir.

In one variant, the autoclave, in particular the unit for sterilizing or partly sterilizing the water before being fed into the chamber, alternatively or additionally includes one or more sterile filters disposed upstream and/or downstream of the instantaneous steam generator. For example, at least one sterile filter is arranged in the feed line from a tank for feed water to the instantaneous steam generator. Alternatively or additionally, at least one sterile filter is arranged in the region of an inlet, in particular in the inlet, to the instantaneous steam generator. In both cases, feed water is sterilized by means of sterile filtration. Alternatively or additionally, at least one sterile filter is arranged in the region of an outlet, in particular in the outlet, of the instantaneous steam generator. In another variant, at least one sterile filter alternatively or additionally is arranged in a feed line from the instantaneous steam generator to the chamber. In the two latter cases, gaseous water is sterilized by means of sterile filtration.

According to one aspect, the autoclave is configured to perform setting of the sterilization condition in the chamber for the duration of the holding time exclusively by means of evaporation of the condensate obtained in the chamber, or the autoclave is configured to feed water into the chamber before the beginning of the holding time and perform setting of the sterilization condition in the chamber for the duration of the holding time exclusively by means of evaporation of the water fed into the chamber before the beginning of the holding time and by means of evaporation of the condensate obtained in the chamber. For this purpose, the autoclave includes a unit for evaporating the condensate obtained in the chamber, or the condensate obtained in the chamber and the water fed into the chamber before the beginning of the holding time. This allows the autoclave to do without a supply of new water, in particular of fresh feed water, to the instantaneous steam generator and thus to the chamber during the holding time. The water used for setting the sterilization condition in the chamber thus is subject to the sterilization condition at least for the duration of the holding time and thus is sterile by the end of the holding time.

In one variant, the autoclave, in particular the unit for evaporating the condensate obtained in the chamber, or for evaporating the condensate obtained in the chamber and the water fed into the chamber before the beginning of the holding time, includes a heating element arranged in the chamber for evaporating the condensate obtained in the chamber, or for evaporating the condensate obtained in the chamber and the water fed into the chamber before the beginning of the holding time. In an alternative variant, the autoclave, in particular the unit for evaporating the condensate obtained in the chamber, or the condensate obtained in the chamber and the water fed into the chamber before the beginning of the holding time, includes the instantaneous steam generator for evaporating the condensate obtained in the chamber, or for evaporating the condensate obtained in the chamber and the water fed into the chamber before the beginning of the holding time, and optionally a pump for supplying condensate, or condensate and water fed into the chamber before the beginning of the holding time, from the chamber into the instantaneous steam generator, wherein the instantaneous steam generator is arranged outside the chamber. The unit for evaporating the condensate obtained in the chamber, or the condensate obtained in the chamber and the water fed into the chamber before the beginning of the holding time, in this variant thus comprises the instantaneous steam generator arranged outside the chamber and optionally a pump for supplying condensate, or condensate obtained in the chamber and water fed into the chamber before the beginning of the holding time, from the chamber into the instantaneous steam generator. In another variant, the autoclave, in particular the unit for evaporating the condensate obtained in the chamber, or the condensate obtained in the chamber and the water fed into the chamber before the beginning of the holding time, includes the instantaneous steam generator for evaporating the condensate obtained in the chamber, or the condensate obtained in the chamber and the water fed into the chamber before the beginning of the holding time, wherein the instantaneous steam generator is arranged in the chamber. The unit for evaporating the condensate obtained in the chamber, or the condensate obtained in the chamber and the water fed into the chamber before the beginning of the holding time, in this variant thus comprises the instantaneous steam generator arranged in the chamber.

### BRIEF DESCRIPTION OF THE DRAWINGS

Details of aspects of the solution claimed here will be explained in detail below with reference to exemplary embodiments and Figures.

FIG. 1 shows a representation of a method for steam sterilization with an autoclave and a corresponding autoclave according to one variant of the proposed solution, wherein all of the water provided in the chamber during the holding time is sterilized after being provided in the chamber.

FIG. 2 shows a representation of a method for steam sterilization with an autoclave and a corresponding autoclave according to an alternative variant of the proposed solution, wherein all of the water provided in the chamber during the holding time is sterilized after being provided in the chamber.

FIG. 3 shows a representation of a method for steam sterilization with an autoclave and a corresponding autoclave according to an alternative variant of the proposed solution, wherein all of the water provided in the chamber during the holding time is sterilized after being provided in the chamber.

FIG. 4 shows a representation of a method for steam sterilization with an autoclave and a corresponding autoclave according to an alternative variant of the proposed solution, wherein all of the water provided in the chamber during the holding time is sterilized after being provided in the chamber.

FIG. 4A shows a representation of a method for steam sterilization with an autoclave and a corresponding autoclave in another variant of the proposed solution.

FIG. 5 shows a representation of a method for steam sterilization with an autoclave and a corresponding autoclave according to a variant of the proposed solution, wherein all of the water provided in the chamber during the holding time is sterilized before being provided in the chamber, or is partly sterilized before being provided in the chamber and is completely sterilized after being provided in the chamber.

FIG. 6 shows a representation of a method for steam sterilization with an autoclave and a corresponding autoclave according to an alternative variant of the proposed solution, wherein all of the water provided in the chamber during the holding time is sterilized before being provided in the chamber, or is partly sterilized before being provided in the chamber and is completely sterilized after being provided in the chamber.

FIG. 7 shows a representation of a method for steam sterilization with an autoclave and a corresponding autoclave according to an alternative variant of the proposed solution, wherein all of the water provided in the chamber during the holding time is sterilized before being provided in the chamber.

FIG. 8 shows a representation of a method for steam sterilization with an autoclave and a corresponding autoclave according to an alternative variant of the proposed solution, wherein all of the water provided in the chamber during the holding time is sterilized before being provided in the chamber.

### DETAILED DESCRIPTION

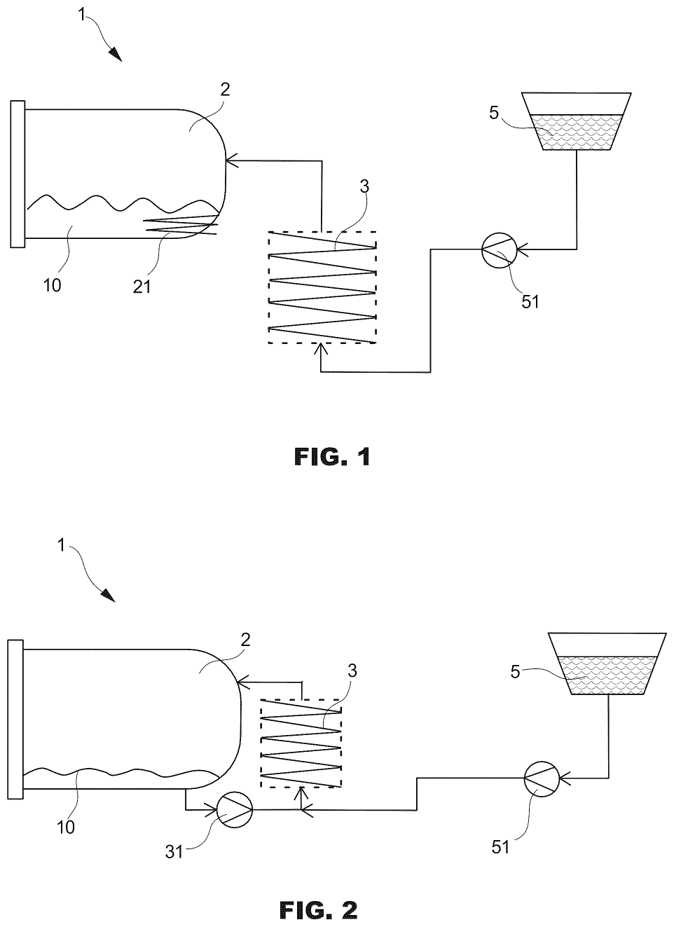

FIG. 1 1 illustrates a method for steam sterilization in an autoclave 1 and a corresponding autoclave 1. In steam sterilization, the number of active, i.e. viable microorganisms, is brought under a normatively defined threshold value. According to DIN EN 556 a medical product for example is regarded as sterile when the theoretical probability of finding a viable microorganism is less than 1:10⁶. This corresponds to an SAL value of 10-6. Microorganisms are rendered harmless during steam sterilization by being exposed to gaseous water, i.e. steam, at a particular sterilization condition, for example to a sterilization temperature at saturated steam conditions over a certain period, the so-called holding time. The holding time depends on the chosen sterilization condition, in particular the sterilization temperature at saturated steam conditions. The standards EN 13060 and EN 285 define, as mentioned already, such relationships between holding time and sterilization conditions. In an autoclave 1, liquid water, so-called feed water, is provided in a tank 5 for generating gaseous water, i.e. steam. The feed water is supplied to a steam generator via a pump 51, as illustrated here. The autoclave 1 described here includes an instantaneous steam generator 3 for generating gaseous water. An instantaneous steam generator 3 provides for a quasi-instantaneous evaporation of the supplied feed water. This renders the instantaneous steam generator 3 attractive for efficiency and cost reasons. During the holding time the gaseous water thus generated, i.e. the steam generated in the instantaneous steam generator 3, is provided in a chamber 2 of the autoclave 1 in order to effect a sterilization of sterile items arranged in the chamber 2. For this purpose, a sterilization condition is set by a corresponding regulating unit of the chamber 2 for the duration of a holding time. For example, a temperature of 121° C. is set at saturated steam conditions for 15 minutes. In use of an instantaneous steam generator 3, the setting of the sterilization condition for the duration of the holding time requires repeated refeeding of gaseous water into the chamber 2. To prevent a contamination of the chamber 2 and of sterile items arranged therein due to refeeding, the autoclave 1 is configured to sterilize all of the water provided in the chamber 2 during the holding time, in particular after being provided in the chamber 2, so that at the end of the holding time exclusively sterile water is present in the chamber 2. In particular, the autoclave 1 is configured to perform the setting of the sterilization condition by means of the regulating unit exclusively by an evaporation of condensate 10 obtained in the chamber 2. In an alternative variant, the autoclave 1 is configured to perform the setting of the sterilization condition by means of the regulating unit exclusively by an evaporation of liquid water fed into the chamber 2 before the beginning of the holding time and by an evaporation of condensate 10 obtained in the chamber 2. In particular, the feeding of water, in particular liquid water, before the beginning of the holding time ensures that during the holding time enough water is available in the chamber 2 in order to set and maintain the sterilization condition. An evaporation and refeeding of fresh feed water from the tank 5 thus can be omitted in both variants. After the beginning of the holding time, no new water is added to the chamber 2. Merely the condensate 10 obtained is newly evaporated. For this purpose, there is provided a unit for evaporating the condensate obtained in the chamber 2, which in the present case comprises a heating element 21 arranged in the chamber 2. The heating element 21 is controlled by means of the regulating unit in such a way that a sterilization condition exists in the chamber 2 for the duration of the holding time. All of the water obtained in the chamber 2 during the holding time thus is subjected to the sterilization condition for the duration of the holding time. Thus, it is sterile by the end of the holding time. There is thus provided a method for steam sterilization in an autoclave 1 and a corresponding autoclave, which combines the advantages of an instantaneous steam generator 3 with a hygienically perfect process management.

FIG. 2 illustrates a method for steam sterilization in an autoclave 1 and a corresponding autoclave 1 according to another variant. In this variant, too, a hygienic process management is achieved in that all of the water provided in the chamber 2 during the holding time is sterilized, in particular after being provided in the chamber 2. Like in the method and the autoclave 1 according to the variant shown in FIG. 1, this likewise is effected in that the sterilization condition during the holding time is set exclusively by evaporation of condensate 10 obtained in the chamber 2 or exclusively by evaporation of water fed into the chamber 2 before the beginning of the holding time and by evaporation of condensate 10 obtained in the chamber 2. For this purpose, there is provided a unit for evaporating the condensate obtained in the chamber 2, in particular the condensate obtained in the chamber and the water fed into the chamber before the beginning of the holding time, which in the present case comprises a pump 31 and the instantaneous steam generator 3 arranged outside the chamber 2. In the variant shown here, in contrast to the variant of FIG. 1, the evaporation of the condensate 10, in particular of the condensate obtained in the chamber and the water fed into the chamber before the beginning of the holding time, is not effected in the chamber 2 itself. Rather, the condensate 10, or the condensate 10 and the water fed into the chamber 2 before the beginning of the holding time, is pumped from the chamber 2 into the instantaneous steam generator 3 for example through a port provided for discharging condensate 10 from the chamber 2. For this purpose, a pump 31 is provided. The condensate 10, or the condensate 10 and the water fed into the chamber 2 before the beginning of the holding time, is evaporated in the instantaneous steam generator 3 and again supplied to the chamber 2. Refeeding fresh feed water to the instantaneous steam generator 3 is not necessary, so that at the end of the holding time all of the water obtained in the chamber 2 during the holding time was subjected to the sterilization condition for the duration of the holding time, and thus is sterile at the end of the holding time. To avoid dead sections and a mixing of condensate 10, or of the condensate 10 and the water fed into the chamber 2 before the beginning of the holding time, with feed water, a non-return valve optionally is provided close to the water circuit formed of tank 5 and pump 51. Alternatively, the pump 51 with an integrated non-return valve is arranged close to the water circuit.

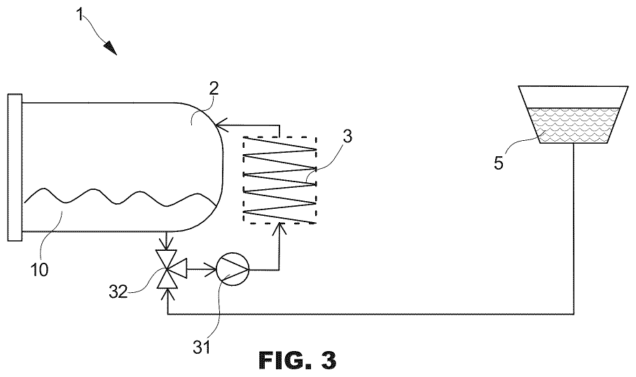

FIG. 3 shows a method for steam sterilization in an autoclave 1 and a corresponding autoclave 1 in a variant slightly modified with respect to the variant of FIG. 2. Thus, in the following, only the modifications with respect to the variant shown with regard to FIG. 2 are described. The variant shown here, as compared to the variant of FIG. 2, does without the pump 51 for feed water. Instead, both the condensate 10 obtained in the chamber 2, or the condensate 10 and the water fed into the chamber 2 before the beginning of the holding time, and feed water supplied before the beginning of the holding time is supplied to a pump 31 and by the same to the instantaneous steam generator 3 via a multi-way valve 32, shown for example as a 3/2-way valve. A supply of feed water from the tank 5 to the instantaneous steam generator 3 is effected merely at the beginning of the holding time. During the holding time, the setting of a sterilization condition is effected merely by the evaporation of condensate 10 obtained in the chamber 2, in particular by evaporation of the condensate obtained in the chamber and the water fed into the chamber before the beginning of the holding time, which by means of the pump 31 is supplied to the instantaneous steam generator 3 and evaporated here. To ensure the sterilization condition for the duration of the holding time, the circuit consisting of the multi-way valve 32, the pump 31 and the instantaneous steam generator 3 for example is configured with short pipe lengths and thermally insulated. In one variant, the holding time is slightly prolonged.

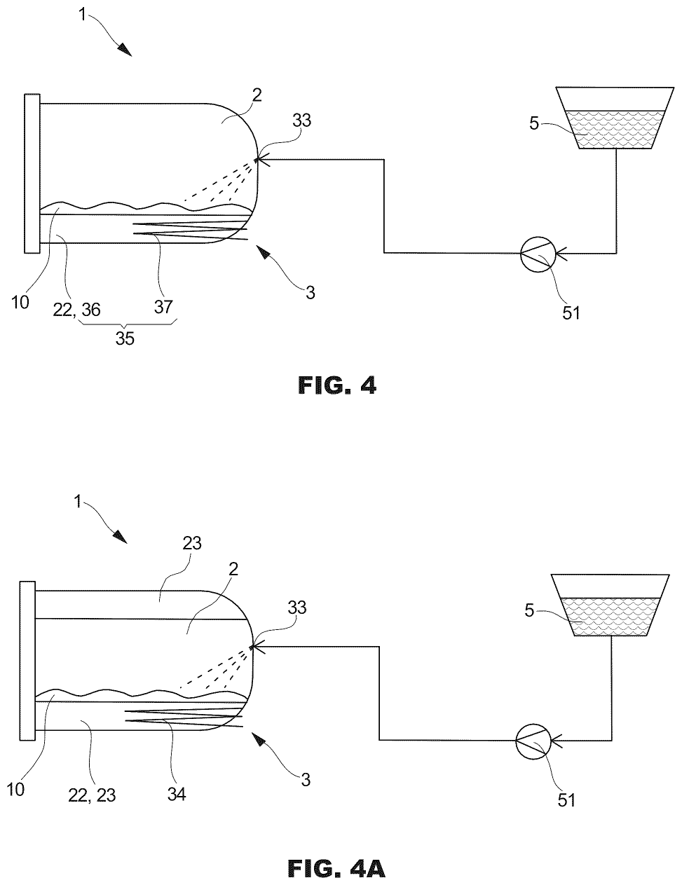

FIG. 4 shows a method for steam sterilization in an autoclave 1 and a corresponding autoclave 1 in another variant. Here as well, all of the water present in the chamber 2 during the holding time is sterilized. In particular, the water is sterilized after being provided in the chamber 2. For this purpose, the instantaneous steam generator 3 is arranged in the chamber 2 so that condensate 10 obtained in the chamber 2, or the condensate 10 obtained in the chamber 2 and the water fed into the chamber 2 before the beginning of the holding time is evaporated in the instantaneous steam generator 3 in the chamber 2 and thus a sterilization condition is set during the holding time in the chamber 2. The instantaneous steam generator 3 comprises a radiator 35 arranged in the chamber 2, in particular a heating coil 37 arranged in a heating block 36 or a heating cartridge (not shown). The heating coil 37 heats the heating block 36. Water, in particular liquid water impinging on the radiator 35 thus is evaporated instantaneously. Liquid water is sprayed into the chamber 2 via a nozzle 33. By means of the nozzle 33, the liquid water is sprayed onto the radiator 35 and evaporated there. The unit for evaporating the condensate obtained in the chamber 2, or the condensate obtained in the chamber and the water fed into the chamber before the beginning of the holding time, thus in the present case comprises the nozzle 33 and the instantaneous steam generator 3 arranged in the chamber 2. The radiator 35 is arranged in the region of the floor 22 of the chamber 2, so that condensate 10 obtained in the chamber 2, or the condensate 10 and the water fed into the chamber 2 before the beginning of the holding time, gets onto the instantaneous steam generator 3 and is evaporated by the same. For example, the heating block 36 forms the floor 22 of the chamber 2. Refeeding of feed water from the tank 5 during the holding time into the chamber 2 or onto the instantaneous steam generator 3 is not effected. All of the water obtained in the chamber 2 during the holding time thus is exposed to the sterilization condition for the duration of the holding time and thus is sterile by the end of the holding time. In this way, a particularly simple construction of an autoclave 1 with instantaneous steam generator 3 and hygienic process management is provided.

In FIG. 4A, a method for steam sterilization with an autoclave 1 and a corresponding autoclave 1 according to the variant shown in a modification of FIG. 4 is explained in more detail. The autoclave 1 includes a chamber 2 and an instantaneous steam generator 3 arranged in the chamber 2. The instantaneous steam generator 3 comprises parts of the chamber wall 23 or the entire chamber wall 23. In particular, the instantaneous steam generator 3 comprises the floor 22 of the chamber 2. In the floor 22 a heating element 34, in particular a heating cartridge, is arranged in the chamber wall 23. The heating element 34, in particular the heating cartridge, heats the chamber wall 23, in particular the chamber floor 22. Water impinging on the chamber wall 23, in particular the chamber floor 22, thus can be evaporated instantaneously. In particular, liquid water is sprayed into the chamber 2 via a nozzle 33. By means of the nozzle 33, the liquid water is sprayed onto a chamber wall 23, in particular a floor 22 of the chamber wall 23, and evaporated there. The unit for evaporating the condensate obtained in the chamber 2, or the condensate obtained in the chamber and the water fed into the chamber before the beginning of the holding time, thus in the present case comprises the nozzle 33 and the instantaneous steam generator 3 arranged in the chamber 2. The chamber 2 itself is an extruded boiler made of a thermally conductive metal, for example an extruded aluminum boiler.

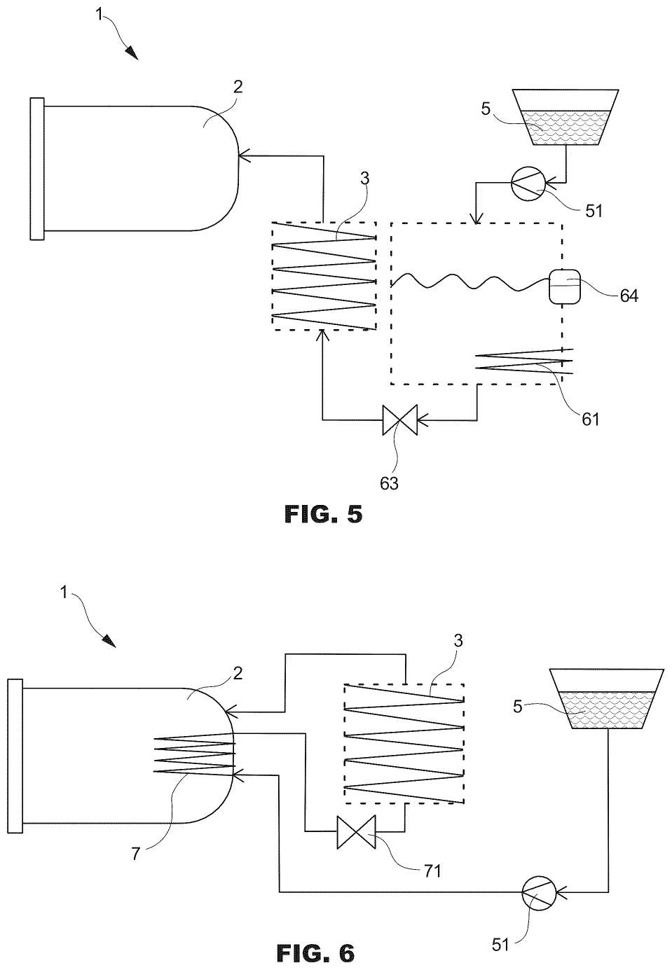

FIG. 5 illustrates a method for steam sterilization in an autoclave 1 and a corresponding autoclave 1 in another variant. In this variant, too, all of the water present in the chamber 2 during the holding time is sterilized. According to the variant shown in FIG. 5, in contrast to the variants shown in FIGS. 1 to 4, all of the water present in the chamber 2 during the holding time is sterilized before being provided in the chamber 2 or is at least partly sterilized before being provided in the chamber 2 and is completely sterilized after being provided in the chamber 2. In the present variant, a sterilization condition in the chamber 2 is set by refeeding gaseous water into the chamber 2 during the holding time, for example by means of the regulating unit. The water is supplied to the instantaneous steam generator 3 for example from a tank 5 for feed water, in particular by means of a pump 51. Before being supplied to the instantaneous steam generator 3, the water is sterilized or partly sterilized. For this purpose, there is provided a unit for sterilizing or partly sterilizing the water before being fed into the chamber 2. In the present case, the same includes a storage tank 6 disposed upstream of the instantaneous steam generator 3. Said storage tank is provided with a heating element 61. By means of the heating element 61, water in the storage tank 6 is heated to a temperature above a sterilization temperature. The sterilization temperature for example is specified by the sterilization condition set in the chamber 2. For example, the water in the storage tank 6 is heated to a temperature of 150° C. The resulting overpressure in one variant is utilized to transfer the preheated water into the instantaneous steam generator 3 by means of a force pilot operated valve 63. The storage tank 6 is configured such that it can store the water volume which is needed for a sterilization program during a holding time. Thus, the water getting into the instantaneous steam generator 3 has already been sterilized, or at least partly sterilized, in the storage tank 6. When merely a partial sterilization of the water is effected in the storage tank 6, the water in the chamber 2 is completely sterilized, for example by being exposed to the sterilization condition in the chamber 2 for the remaining duration of the holding time. In the venting and steam inlet phases preceding the holding time, a temperature below the sterilization temperature can exist in the storage tank 6. In these phases, the feed water from the tank 5 is passed through the storage tank 6 into the instantaneous steam generator 3 by means of the pump 51 or guided through the storage tank 6 into the instantaneous steam generator 3 by means of an overpressure existing in said storage tank. To avoid a dead section and a risk of mixing of water present in the storage tank with feed water originating from the tank 5, a non-return valve optionally is arranged in the region of the inlet of the storage tank 6.

FIG. 6 shows a method for steam sterilization in an autoclave 1 and a corresponding autoclave 1 in another variant. In this variant, too, like in the variant described with respect to FIG. 5, the water is sterilized or partly sterilized before being supplied to the instantaneous steam generator 3. For this purpose, there is provided a unit for sterilizing or partly sterilizing the water before being fed into the chamber 2. In the present case, the same includes a coil 7 arranged in the chamber 2, which is disposed upstream of the instantaneous steam generator 3. In an alternative variant, the coil 7 is arranged on the chamber 2. It is important that the water flowing through the coil is subjected to the sterilization condition in the chamber 2 for the duration of the holding time. The coil is configured such that it can store a water volume needed for a sterilization process during the holding time. In this variant, fresh feed water likewise is supplied to the instantaneous steam generator 3 for setting a sterilization condition in the chamber 2, but in this variant, too, the water is already sterilized or at least partly sterilized when it reaches the instantaneous steam generator 3. When the water in the coil 7 merely is partly sterilized, for example because a sterilization condition in the coil 7 is not reached for the duration of the holding time, a complete sterilization is effected in the chamber 2. The feeding operation from the tank 5 is effected by means of a pump 51 and a force pilot operated valve 71 arranged between coil 7 and instantaneous steam generator 3. When the autoclave 1 is operated in a pure overpressure method, the valve 71 can be omitted. To avoid a dead section and a risk of mixing of water present in the coil with feed water from the tank 5, a non-return valve in one variant is arranged in the region of the inlet of the coil 7. In this variant, too, an autoclave 1 comprising an instantaneous steam generator 3 is hygienically operated.

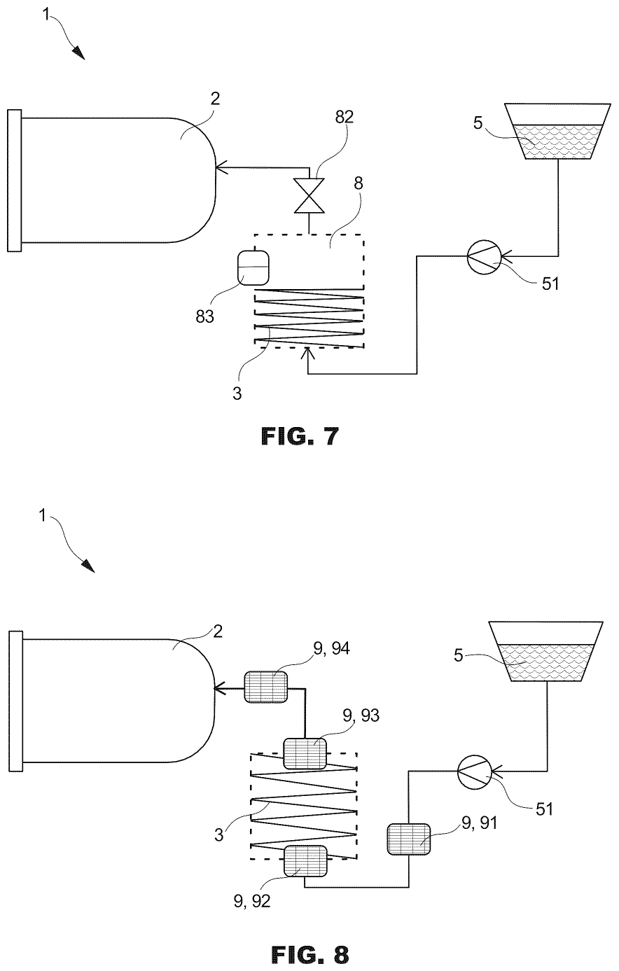

FIG. 7 illustrates a method for steam sterilization in an autoclave 1 and a corresponding autoclave 1 in another variant. In this variant, all of the water provided in the chamber 2 during the holding time is sterilized before being provided in the chamber 2. At the same time, setting of the sterilization condition in the chamber 2 during the holding time also is effected by refeeding gaseous water into the chamber 2. For this purpose, there is provided a unit for sterilizing or partly sterilizing the water before being fed into the chamber 2. The same comprises the instantaneous steam generator 3 with a steam reservoir 8 sealed off with respect to the chamber 2. Gaseous water generated in the instantaneous steam generator 3 thus can be stored in the steam reservoir 8 and be passed into the chamber 2 for setting the sterilization condition during the holding time. The illustrated variant thus represents a mixed form of a classical instantaneous steam generator 3 and a sealed-off steam generator. The steam reservoir 8 is sealed off from the chamber 2 for example by means of a valve 82. In the instantaneous steam generator 3 gaseous water for example is generated during a venting and heating phase and stored in the steam reservoir 8. During the holding time, gaseous water is passed from the steam reservoir 8 into the chamber 2 for setting the sterilization condition in the chamber 2 by adjusting operations at the valve 82. During the holding time, no fresh feed water thus is passed from the tank 5 into the instantaneous steam generator 3 and thus into the chamber 2. All of the water provided in the chamber 2 during the holding time was sterilized in the steam reservoir 8, i.e. before being provided in the chamber 2. This provides for a hygienic steam sterilization in an autoclave 1 with instantaneous steam generator 3. To avoid a dead section and a risk of mixing of water present in the instantaneous steam generator 3 with feed water from the tank 5, a non-return valve in one variant is arranged at the inlet to the instantaneous steam generator 3.

FIG. 8 shows a method for steam sterilization in an autoclave 1 and a corresponding autoclave 1 in another variant. In this variant, all of the water provided in the chamber 2 during the holding time is sterilized in the chamber 2 before being provided. Setting of a sterilization condition in the chamber 2 also is effected by refeeding feed water from the tank 5 into the instantaneous steam generator 3. The unit for sterilizing or partly sterilizing the water before feeding the same into the chamber 2 in the present case comprises at least one sterile filter 9. The sterilization before provision in the chamber 2 is effected by at least one sterile filter 9. Depending on the variant, one or more sterile filters 9 are provided. One or more of the sterile filters 91 to 94 shown in this FIG. 8 can be arranged in a variant of the autoclave 1. For example, before being supplied to the instantaneous steam generator 3 the water, in particular the liquid water, is sterilized by means of at least one sterile filter 91, 92. At least one sterile filter 91 is arranged for example in a feed line for feed water from the tank 5 to the instantaneous steam generator 3. As such a sterile filter 91 does not benefit from the thermal performance of the instantaneous steam generator 3, it must be exchanged when reaching its hygienic capacity. Alternatively or additionally, at least one sterile filter 92 is arranged in the region of an inlet, in particular in the inlet, of the instantaneous steam generator 3. Such a sterile filter 92 benefits from the heat emitted by the instantaneous steam generator 3 and is thermally sterilized continuously by the same so that an exchange of the sterile filter 92 becomes necessary only at the end of the technical lifetime of the sterile filter 92. Alternatively or additionally at least one sterile filter 93, 94 is provided, by means of which the gaseous water generated in the instantaneous steam generator 3 is sterilized before being provided in the chamber 2. For example, a sterile filter 93 is arranged in the region of the outlet of the instantaneous steam generator 3, in particular in the outlet of the instantaneous steam generator 3. Such a sterile filter 93 is continuously thermally sterilized by the action of heat of the instantaneous steam generator 3. In addition, the gaseous water flowing through the same is sterilized. As the sterile filter 93 also holds up any aerosol droplets remaining in the gaseous water, the quality of the gaseous water is improved by the sterile filter 93 not only hygienically, but also technically. The same applies for at least one sterile filter 94 alternatively or additionally arranged in the feed line between instantaneous steam generator 3 and chamber 2. Due to the throughflow with gaseous water, such a sterile filter 94 is sterilized continuously and likewise improves the technical quality of the gaseous water before the same is supplied into the chamber 2. With this variant, too, a hygienic steam sterilization with instantaneous steam generation becomes possible.

### LIST OF REFERENCE NUMERALS

1 autoclave
2 chamber
21 heating element
22 chamber floor
23 chamber wall
3 instantaneous steam generator
31 pump
32 multi-way valve
33 nozzle
34 heating element
35 radiator
36 heating block
37 heating coil
5 feed water tank
51 pump
6 storage tank
61 heating element
63 valve
64 level regulation
7 coil
71 valve
8 steam reservoir
82 valve
83 level regulation
9 sterilization filter
91 sterilization filter
92 sterilization filter
93 sterilization filter
94 sterilization filter
10 condensate

1. A method for steam sterilization in an autoclave, the method comprising:
generating gaseous water in an instantaneous steam generator,
providing the gaseous water in a chamber of the autoclave for sterilizing sterile items arranged in the chamber,
setting a sterilization condition in the chamber for the duration of a holding time,
wherein all of the water present in the chamber during the holding time is sterilized.

2. The method according to claim 1, wherein all of the water present in the chamber during the holding time is sterilized before being provided in the chamber or is sterilized after being provided in the chamber or is partly sterilized before being provided in the chamber and completely sterilized after being provided in the chamber.

3. The method according to claim 1, wherein setting of the sterilization condition during the holding time is effected exclusively by evaporating condensate obtained in the chamber or that water is fed into the chamber before the beginning of the holding time and setting of the sterilization condition during the holding time is effected exclusively by evaporating the water fed into the chamber before the holding time and by evaporating condensate obtained in the chamber.

4. The method according to claim 1, wherein a condensate obtained in the chamber, or condensate obtained in the chamber and water fed into the chamber before the beginning of the holding time is evaporated in the chamber, wherein the evaporation is effected by means of a heating element arranged in the chamber or wherein the evaporation is effected by means of the instantaneous steam generator, wherein the instantaneous steam generator is arranged in the chamber.

5. The method according to claim 1, wherein a condensate obtained in the chamber, or condensate obtained in the chamber and water fed into the chamber before the beginning of the holding time is evaporated outside the chamber, wherein the evaporation is effected by means of the instantaneous steam generator, wherein the instantaneous steam generator is arranged outside the chamber.

6. The method according to claim 5, wherein the condensate obtained in the chamber, or the condensate obtained in the chamber and the water fed into the chamber before the beginning of the holding time, is pumped from the chamber into the instantaneous steam generator arranged outside the chamber by means of a pump.

7. The method according to claim 1, wherein setting of the sterilization condition during the holding time also is effected by refeeding gaseous water into the chamber.

8. The method according to claim 7, wherein the instantaneous steam generator includes a steam reservoir sealed off with respect to the chamber by means of a valve, and in the instantaneous steam generator gaseous water is generated in stock and passed into the chamber during the holding time for setting the sterilization condition.

9. The method according to claim 7, wherein the water is sterilized before being supplied to the instantaneous steam generator.

10. The method according to claim 9, wherein the sterilization of the water in a storage tank disposed upstream of the instantaneous steam generator is effected by heating to a temperature above a sterilization temperature or that the sterilization of the water is effected in a coil arranged in or on the chamber, wherein the coil stores a water volume needed for the sterilization process during the holding time; or that the water is sterilized by means of a sterile filter before being supplied to the instantaneous steam generator and/or the gaseous water generated in the instantaneous steam generator is sterilized by means of a sterile filter before being provided in the chamber.

11. An autoclave for sterilizing sterile items, the autoclave including:
an instantaneous steam generator for generating gaseous water;
a regulating unit for setting a sterilization condition for sterilizing sterile items in a chamber of the autoclave for the duration of a holding time;
wherein the autoclave is configured to sterilize all of the water present in the chamber during the holding time, so that at the end of the holding time exclusively sterile water is present in the chamber.

12. The autoclave according to claim 11, wherein the autoclave is configured to sterilize all of the water present in the chamber during the holding time before being provided in the chamber or to sterilize the water after being provided in the chamber or to partly sterilize the water before being provided in the chamber and completely sterilize the water after being provided in the chamber.

13. The autoclave according to claim 11, wherein the instantaneous steam generator is arranged outside the chamber and the autoclave includes a heated storage tank disposed upstream of the instantaneous steam generator for heating water to a temperature above a sterilization temperature, or the autoclave includes a coil arranged in the chamber, which is disposed upstream of the instantaneous steam generator, for storing a water volume needed for a sterilization process, or the autoclave includes a steam reservoir of the instantaneous steam generator sealed off with respect to the chamber by means of a valve, or the autoclave includes one or more sterile filters disposed upstream and/or downstream of the instantaneous steam generator.

14. The autoclave according to claim 11, wherein the autoclave is configured to perform setting of the sterilization condition in the chamber for the duration of the holding time exclusively by means of evaporation of the condensate obtained in the chamber, or the autoclave is configured to feed liquid water into the chamber before the beginning of the holding time and perform setting of the sterilization condition in the chamber for the duration of the holding time exclusively by means of evaporation of the water fed into the chamber before the beginning of the holding time and by means of evaporation of the condensate obtained in the chamber.

15. The autoclave according to claim 14, wherein for evaporating the condensate obtained in the chamber, or for evaporating the condensate obtained in the chamber and the water fed into the chamber before the beginning of the holding time the autoclave includes a heating element arranged in the chamber, or that for evaporating the condensate obtained in the chamber, or for evaporating the condensate obtained in the chamber and the water fed into the chamber before the beginning of the holding time the autoclave includes the instantaneous steam generator and a pump for supplying condensate from the chamber into the instantaneous steam generator, wherein the instantaneous steam generator is arranged outside the chamber, or that for evaporating the condensate obtained in the chamber, or for evaporating the condensate obtained in the chamber and the water fed into the chamber before the beginning of the holding time, the autoclave includes the instantaneous steam generator, wherein the instantaneous steam generator is arranged in the chamber.
